(19) United States
(12) Patent Application Publication
(10) Pub. No.: US 2026/0272680 A1
(43) Pub. Date: Sep. 17, 2026

(54) DEVICES FOR SETTING THICKNESS DURING TOTAL KNEE ARTHROPLASTY

(71) Applicants: Joseph B. ASSINI, Denver, CO (US); Alexander P. SAH, Denver, CO (US)

(72) Inventors: Joseph B. ASSINI, Denver, CO (US); Alexander P. SAH, Denver, CO (US)

(21) Appl. No.: 19/470,792

(22) PCT Filed: Mar. 27, 2024
(86) PCT No.: PCT/US2024/021820

§ 371 Date: Sep. 29, 2025

Related U.S. Application Data
(60) Provisional application No. 63/492,472, filed on Mar. 27, 2023.

(51) Int. Cl. A61F 2/46; A61F 2/30; A61F 2/38

(52) U.S. Cl. CPC A61F 2/4684; A61F 2/3859; A61F 2/389; A61F 2002/30556; A61F 2002/30601

(57) ABSTRACT

Described herein are mechanical, self-contained, non-implantable devices for setting polyethylene thickness during intra-operative trialing and balancing of a total knee arthroplasty. These adjustable trialing tibial insert apparatus may be used with a total knee arthroplasty (TKA) system in order to optimally set the thickness and/or other dimensions of the articular surface of the TKA. These adjustable trialing tibial insert apparatus may include a plurality of pre-set thickness settings, where the thickness is the thickness between a femoral articular surface and the tibial seating surface of the adjustable trialing tibial insert apparatus.

### CLAIM OF PRIORITY

This patent application claims priority to U.S. Provisional Patent Application No. 63/492,472, titled “DEVICES FOR SETTING POLYETHYLENE THICKNESS DURING TOTAL KNEE ARTHROPLASTY” and filed on Mar. 27, 2023, herein incorporated by reference in its entirety.

### BACKGROUND

With the growth of total knee arthroplasty (TKA) and increasing demand for the procedure in both hospital and outpatient settings, operating room efficiency is of utmost importance. During total knee primary and revision replacement procedures the surgeon must decide on the thickness of polyethylene insert that best recreates the function of the knee in terms of motion and balance. The many size options of the femoral and tibial components in knee arthroplasty demands a large inventory of trial tibial articular surfaces to be available during each procedure. Stocking and cleaning of these individual insert trials is costly and space consuming. Furthermore, changing and trialing various poly insert sizes during surgery is a time consuming and challenging part of the procedure.

Currently, all major TKA designs consist of a femoral and a tibial component. Each component is sized to match the patient's anatomy and is secured to the bone, and a plastic (polyethylene) implant (aka tibial insert and/or articular surface) fits between the femoral and tibial components. This tibial insert (“implant”) typically comes in many different thicknesses and is a component of all modern TKA system designs. This implant is typically formulated in polyethylene. Different thicknesses of trial implants (“trials”) are inserted, tested, and removed during the operation to judge knee soft tissue balance and knee motion. Once the final thickness has been selected, the final implantable poly of desired thickness is placed and locked to the tibial component as per the manufacturer design. However, removing and replacing the trial articular surfaces multiple times during the trialing phase of TKA can add significant time and frustration to the operative procedure. What is needed are methods and apparatuses (e.g., devices) that may address these problems.

### SUMMARY OF THE DISCLOSURE

Described herein are mechanical, self-contained, non-implantable (e.g., inserted and removed during the procedure) apparatuses, e.g., devices and systems, and methods for setting articular surface thickness during intra-operative trialing and balancing of Total Knee Arthroplasty (TKA). These apparatuses may be referred to as adjustable trialing tibial inserts, or simply adjusting inserts, for convenience. The adjustable trialing tibial inserts described herein may be used as part of a primary or revision TKA to adjust or select the thickness for a tibial insert, to determine the optimal thickness of the chosen tibial insert, e.g., a polyethylene insert. The ability to adjust the height (thickness) in order to optimize the dimensions of a tibial insert using a self-contained mechanism (e.g., ratcheting, rotational, magnetic, mechanical etc.) that expands the tibial trial thickness would overcome many challenges in implant selection and sizing in TKA. Described herein are selectably expandable tibial insert/articular surfaces.

Existing trial tibial inserts are temporary components used in TKA to assess stability, range of motion, and soft tissue balancing. However, current trialing systems typically require a variety of different trial inserts having different dimensions, that must be inserted and removed multiple times. Further, there is recent evidence showing that trial tibial inserts do not accurately reproduce the same joint kinematics as final inserts. The adjustable trialing tibial insert described herein may provide a single adjustable trialing tibial insert that may be incrementally adjustable by the user (e.g., medical practitioner, surgeon, etc.) after a single insertion, to adjust the distraction of the joint in a manner that allows for more realistic comparison with the final implant.

In general, the adjustable trialing tibial inserts and methods described here may be configured to allow the user to easily see the current or selected thickness (e.g., height) of the adjustable trialing tibial insert, and to readily select a different thickness. In addition, the adjustable trialing tibial inserts described herein are configured to reproduce the mechanical properties of the final implant at each selected thickness. The adjustable trialing tibial inserts have a preset plurality of (e.g., 2 or more, 3 or more, 4 or more, etc.) thickness corresponding to the final implant that will be inserted. Further, the adjustable trialing tibial inserts include one or more stabilizing features that allow the adjustable trialing tibial insert to reproduce the mechanical stability of the implant even at different preset thicknesses that may be selected. These apparatuses may include features that dramatically improve the reliability and effectiveness of the adjustable trialing tibial insert, including in testing (e.g., flexion/extension cycling, torsion, and laxity testing) during the implantation procedure. This may be particularly challenging for an expandable insert, as the effect of forces acting on the implant (e.g., during flexion and extension cycling) may vary as the height varies. The adjustable trialing tibial inserts described herein may be configured to have stability at each of the selectable pre-set heights.

In a general, described herein are mechanically adjustable devices (e.g., adjustable by a ratchet, cam, magnetic, electric, or other mechanical technique) built into the adjustable trialing tibial inserts. The thickness of the adjustable trialing tibial inserts may be increased incrementally (e.g., with each ‘click’ or ‘turn’ or ‘movement’ of the mechanical device) to a variety of pre-set thicknesses. These discrete sizes may be selected to be physiologically relevant, and may correspond to the dimensions of the final articulating surface liner (implant) to be inserted. This alleviates the necessity of removing a trial insert repeatedly and re-inserting the next thicker or thinner size. Instead, the surgeon may simply adjust through the common thicknesses of this self-contained and adjustable trialing tibial insert and determine which appropriate tibial articular surface thickness and then select the final polyethylene liner (implant) for insertion.

In general, the apparatuses described herein may have an articular side (superiorly) which may match to the femur and a component side (inferiorly) which may match the tibial component. In this case, modular articular or component adapters, single or both, may be placed on the adjustable unit. Alternatively the mechanical device may be contained within a completely self-contained trial. Both concepts satisfy the objective of balancing the flexion and extension space and balancing the knee without having to remove and reinsert multiple pieces to achieve different component thicknesses.

In some examples, one mechanical device may be used per polyethylene component size.

In general, the adjustable trialing tibial inserts described herein are not implanted for long periods of time, but are inserted acutely for sizing/testing, and may have a modular design, simplifying sterilization. The apparatuses and methods described herein may be used across multiple different TKA implant designs in both primary and revision settings. That is, the adjustable trialing tibial inserts described herein may allow thickness adjustment and may be matched or built into multiple different poly shapes/models including different designs, TKA philosophy and TKA/poly constraints.

For example, described herein are adjustable trialing tibial insert apparatus for setting articular surface thickness during a total knee arthroplasty (TKA) or revision procedure, the apparatus comprising: a femoral engagement plate having a femoral articular surface that is configured to engage with a femoral component of a TKA system; a tibial plate engagement frame having a tibial seating surface that is configured to engage with a tibial plate of the TKA system; a distraction selector cam having a cam surface, the cam surface a plurality of positions comprising a plurality of pre-set distraction thickness between the femoral articular surface and the tibial seating surface; and a distraction input selector coupled to the distraction selector cam and configured to be actuated to drive movement of the distraction selector cam to change a distraction thickness between the femoral articular surface and the tibial seating surface.

In some cases the adjustable trialing tibial insert apparatus for setting articular surface thickness during a total knee arthroplasty (TKA) or revision procedure comprises: a femoral engagement plate having a femoral articular surface that is configured to engage with a femoral component of a TKA system; a tibial plate engagement frame having a tibial seating surface that is configured to engage with a tibial plate of the TKA system; a distraction selector cam having a cam surface, the distraction selector cam comprising a plurality of flattened sides on the cam surface each corresponding to a pre-set distraction thickness between the femoral articular surface and the tibial seating surface; one or more distraction selector cam follower surfaces configured to support the cam surface of the distraction selector cam; and a distraction input selector coupled to the distraction selector cam and configured to be actuated to drive movement of the distraction selector cam against the one or more distraction selector cam follower surfaces to change a distraction thickness between the femoral articular surface and the tibial seating surface; wherein the femoral engagement plate and the tibial plate engagement frame are coupled together via one or more telescoping supports configured to provide lateral support and prevent rocking at each of the preset distraction thicknesses corresponding to the plurality of flattened sides on the cam surface.

An adjustable trialing tibial insert apparatus for setting articular surface thickness during a total knee arthroplasty (TKA) or revision procedure may include: a femoral engagement plate having a femoral articular surface that is configured to engage with a femoral component of a TKA system; a tibial plate engagement frame having a tibial seating surface that is configured to engage with a tibial plate of the TKA system; a distraction selector cam having a cam surface, the distraction selector cam comprising a plurality of flattened sides on the cam surface each corresponding to a pre-set distraction thickness between the femoral articular surface and the tibial seating surface; one or more distraction selector cam follower surfaces configured to support the cam surface of the distraction selector cam; a visualization window through an anterior region of the femoral engagement plate, wherein the visualization window is configured allow visualization of a distraction selector output indicator on the distraction selector cam indicating a current distraction thickness between the femoral articular surface and the tibial seating surface; a distraction input selector configured to be actuated to drive movement of the distraction selector cam to change the current distraction thickness between the femoral articular surface and the tibial seating surface, wherein the femoral engagement plate and the tibial plate engagement frame are coupled together via one or more telescoping supports configured to provide lateral support between the femoral articular surface and the tibial seating surface at each of the preset distraction thicknesses.

In any of these apparatuses, the cam surface may comprise a plurality of flattened sides each corresponding to a pre-set distraction thickness of the plurality of pre-set distraction thicknesses between the femoral articular surface and the tibial seating surface.

Any of these apparatuses may include a visualization window through the femoral engagement plate, wherein the visualization window is configured allow visualization of a distraction selector output indicator indicating a current distraction thickness between the femoral articular surface and the tibial seating surface. The window may be open (uncovered) or covered by a transparent material. In any of these examples, the visualization window is on a central anterior region of the femoral engagement plate.

In any of these apparatuses, the distraction selector output indicator may indicate the pre-set distraction thickness between the femoral articular surface and the tibial seating surface is on the distraction selector cam. In some cases the plurality of pre-set distraction thicknesses corresponds to thickness between 8 mm and 16 mm. For example, the plurality of pre-set distraction thicknesses may include at least: 9 mm, 11 mm, and 13 mm. In some cases the plurality of pre-set distraction thicknesses may include at least: 10 mm, 12 mm and 14 mm.

Any of these apparatuses may include one or more selector cam follower surfaces. The one or more selector cam follower surfaces may be on the tibial seating surface in some examples. The distraction input selector may be on an anterior region of the apparatus. The femoral engagement plate and the tibial plate engagement frame may be coupled together via one or more telescoping supports configured to provide lateral support and prevent rocking between the femoral articular surface and the tibial seating surface at each of the preset distraction thicknesses corresponding to the plurality of flattened sides on the cam surface. For example, the one or more telescoping supports comprises a plurality of telescoping supports. The one or more telescoping supports may comprise a plurality of bilateral telescoping supports.

Any of these apparatuses may include a posterior cam member coupled to the distraction selector cam via the distraction input selector, wherein activation of the distraction input selector changes the distance between the posterior cam member and the distraction selector cam to change the distraction thickness between the femoral articular surface and the tibial seating surface.

Also described herein are methods of using any of these apparatuses, including using them as part of a TKA procedure. For example, a method of determining an articular surface thickness for a total knee arthroplasty (TKA) or revision procedure using an adjustable trialing tibial insert apparatus may include: inserting an adjustable trialing tibial insert apparatus between a femoral component and tibial plate of a TKA system, wherein the adjustable trialing tibial insert is in a first thickness configuration; checking a response of the knee to a load and/or movement corresponding to the first thickness configuration; changing the thickness of the adjustable trialing tibial insert apparatus from the first thickness configuration to a second, pre-set, thickness configuration; checking a response of the knee to a second load and/or movement corresponding to the second, pre-set, thickness configuration; and removing the adjustable trialing tibial insert apparatus and selecting an articular surface to be positioned between the femoral component and tibial plate of the TKA system based on the responses of the knee to the load and/or movement.

Any of these methods may include repeating the steps of changing the thickness of the adjustable trialing tibial insert apparatus and checking the response of the knee before removing the adjustable trialing tibial insert. Checking the response of the knee to the load and/or movement may comprise checking the response of the knee to one or more of: a plurality of flexion and extension cycles and a torsional/twisting movement. Changing the thickness of the adjustable trialing tibial insert apparatus from the first thickness configuration to the second, pre-set, thickness configuration may comprise activating a distraction input selector to drive movement of a distraction selector cam against one or more distraction selector cam follower surfaces to change a distraction thickness between a femoral articular surface and a tibial seating surface of the adjustable trialing tibial insert apparatus. Changing the thickness of the adjustable trailing tibial insert apparatus may comprise rotating the distraction selector cam so that one of a plurality of flattened sides on the cam surface rests against the one or more distraction selector cam follower surfaces, wherein each of flattened side of the plurality of flattened sides corresponds to a pre-set distraction thickness between the femoral articular surface and the tibial seating surface.

Any of these methods may include visually observing a distraction selector output indicator through a visualization window of a femoral articular surface of the adjustable trialing tibial insert apparatus.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIGS. 1A-1B illustrate an examples of a total knee arthroplasty (TKA) system including a femoral component, a stemmed tibial component (tibial plate) and an articulating surface implant. FIG. 1B shows the PKA system of claim 1 implanted into a knee.

FIGS. 2A-2C schematically illustrates an example of an adjustable trialing tibial insert apparatus. FIG. 2A shows the adjustable trialing tibial insert apparatus. FIGS. 2B-2C illustrate application of the adjustable trialing tibial insert apparatus of FIG. 2A on a tibial component of a TKA system.

FIGS. 3A-3B show front (anterior) perspective and back (posterior) perspective exploded views, respectively, of the adjustable trialing tibial insert apparatus of FIGS. 2A-2C.

FIGS. 4A-4F illustrate distraction of an adjustable trialing tibial insert apparatus similar to that of FIGS. 2A-2C and 3A-3B. FIGS. 4A-4B show front perspective and side views, respectively, of the adjustable trialing tibial insert apparatus in the most compact configuration (e.g., 10 mm). FIGS. 4C and 4D show front perspective and side views, respectively, of the adjustable trialing tibial insert apparatus in a first pre-set thickness (e.g., 12 mm) configuration. FIGS. 4E and 4F show front perspective and side views, respectively, of the adjustable trialing tibial insert apparatus in as second pre-set thickness (e.g., 14 mm) configuration.

FIGS. 5A-5C show side sectional views through an adjustable trialing tibial insert apparatus similar to that shown in FIGS. 4A-4F, illustrating the change in distraction (thickness) of the femoral articular surface of the adjustable trialing tibial insert apparatus relative to the tibial seating surface of the adjustable trialing tibial insert apparatus.

FIGS. 6A-6B illustrate a second example of an adjustable trialing tibial insert apparatus described herein.

FIGS. 7A-7B schematically illustrate another example of an adjustable trialing tibial insert apparatus described herein. FIG. 7A shows a side perspective view and FIG. 7B shows a partially transparent view.

FIGS. 8A-8F show additional views of the adjustable trialing tibial insert apparatus of FIGS. 7A-7B. FIG. 8A shows a section through the anterior-to-posterior axis (midline) of the adjustable trialing tibial insert apparatus. FIG. 8B shows an exploded view. FIGS. 8C and 8D show bottom perspective views of a portion of the adjustable trialing tibial insert apparatus. FIG. 8E shows a partially exploded view of the adjustable trialing tibial insert apparatus. FIG. 8F shows a section through the midline of the adjustable trialing tibial insert apparatus.

FIGS. 9A-9L illustrate distraction (e.g., controllably setting the thickness) of an example of an adjustable trialing tibial insert apparatus described herein. FIGS. 9A, 9B and 9C show the adjustable trialing tibial insert apparatus in a first thickness configuration (e.g., 10 mm separation between the femoral articular surface and the tibial seating surface), in a side perspective, side and sectioned views, respectively. FIGS. 9D, 9E and 9F show the adjustable trialing tibial insert apparatus in a second thickness configuration (e.g., 12 mm separation between the femoral articular surface and the tibial seating surface), in a side perspective, side and sectioned views, respectively. FIGS. 9G, 9H and 9I show the adjustable trialing tibial insert apparatus in a third thickness configuration (e.g., 14 mm separation between the femoral articular surface and the tibial seating surface), in aside perspective, side and sectioned views, respectively. And FIGS. 9J, 9K and 9L show the adjustable trialing tibial insert apparatus in a fourth thickness configuration (e.g., 16 mm separation between the femoral articular surface and the tibial seating surface), in a side perspective, side and sectioned views, respectively.

FIGS. 10A-10F schematically illustrate another example of an adjustable trialing tibial insert apparatus as described herein. FIG. 10A shows a side perspective view, FIG. 10B shows an anterior view, FIG. 10C shows an exploded view, FIG. 10D shows an anterior perspective view, and FIGS. 10E-10F show section views through a midline of the adjustable trialing tibial insert apparatus.

FIG. 11 schematically illustrates one example of a method of using an adjustable trialing tibial insert apparatus as described herein.

### DETAILED DESCRIPTION

Described herein are adjustable trialing tibial insert apparatus that may be used with a total knee arthroplasty (TKA) system in order to optimally set the thickness and/or other dimensions of the articular surface of the TKA. These apparatuses may include a plurality of pre-set thickness settings, where the thickness is the thickness between a femoral articular surface and the tibial seating surface of the adjustable trialing tibial insert apparatus.

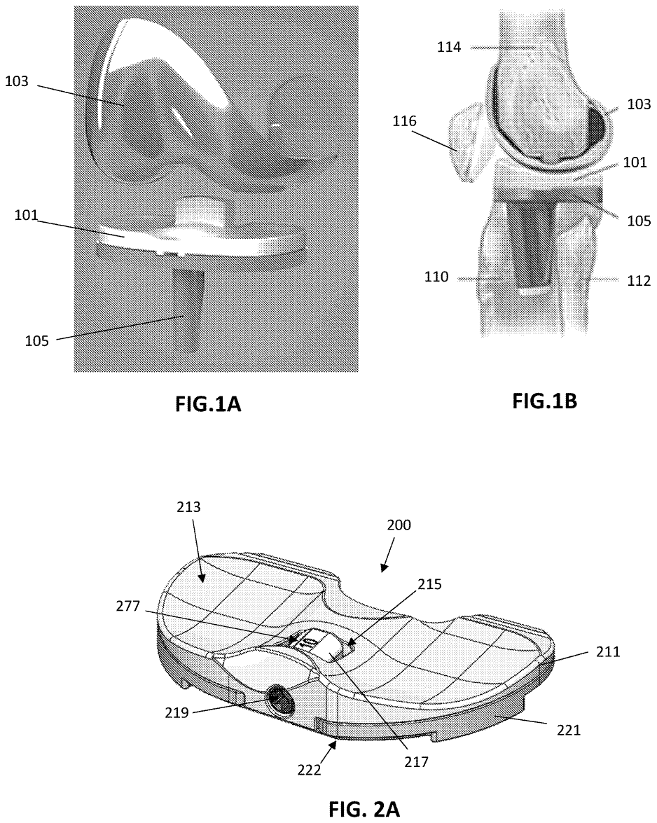

A total knee arthroplasty (TKA) system typically consists of a femoral component and a tibial component (e.g., tibial plate) that are implanted into the bone. This is illustrated in FIGS. 1A-1B. Each component is secured to the bone and a plastic (e.g., polyethylene) implant (also referred to as a tibial insert or articular surface) fits between the femoral and tibial components. In FIGS. 1A-1B a total knee implant including a femoral component 103, a stemmed tibial plate 105 and a polyethylene tibial insert 101 are shown. FIG. 1B shows the TKA system of FIG. 1A coupled to the femur 114 and tibial 110. The patella 116 and fibula 112 are shown for completeness. The tibial insert (implant 101) typically comes in many thicknesses and is selected by the user (e.g., surgeon). This component is typically formulated in polyethylene. During a surgical procedure, the user must test different thicknesses of trial articular surfaces (“trials”) before selecting the final implant to insert, by individually inserting each trial and judging knee soft tissue balance and knee motion. Once the final thickness has been selected, the final implant having the desired thickness is placed and locked to the tibial component as per the manufacturer design. As mentioned, the process of trialing may require removing and replacing the trials (e.g., the trial articular surfaces) multiple times and can add significant time and frustration to the operative procedure.

In general, an adjustable trialing tibial insert apparatus may include a mechanical actuator and may be positioned between the articular side and the component side of the tibial insert. The thickness of the insert may be selected by the user (e.g., physician) in order to determine the appropriate size of the articular surface to be implanted.

In any of these apparatuses, the adjustable trialing tibial insert apparatus may be configured for setting articular surface thickness during a TKA or revision procedure. These apparatuses may be modular, including a femoral engagement plate that may be moved between pre-set positions (or, less desirably, in some cases continuous positions) relative to a tibial plate engagement frame. The femoral engagement plate may include a femoral articular surface that is configured to engage with a femoral component of a TKA system. The tibial plate engagement frame may have a tibial seating surface that is configured to engage with a tibial plate of the TKA system. These upper and lower surface of the apparatus may be configured to match the general profile of the articular surface implant 101 (e.g., the polyethylene implant) that is to be more permanently implanted between the femoral component and the tibial plate of the TKA.

In general, these apparatuses may include a distractor mechanism that is configured to change the distance (thickness) between the femoral articular surface and the tibial seating surface. For example, any of these apparatuses may include a distraction selector cam having one or more cam surfaces. Movement of the distraction selector cam may drive the cam surface against a follower surface (e.g., a distractor selector cam follower) to increase the separation between the femoral articular surface and the tibial seating surface. In general, the cam surface may have a plurality of positions each corresponding to a pre-set distraction thickness between the femoral articular surface and the tibial seating surface. The distraction selector may be moved, and the position selected by, a distraction input selector that is coupled to the distraction selector cam and configured to be actuated to drive movement of the distraction selector cam to change a distraction thickness between the femoral articular surface and the tibial seating surface.

Any of these apparatuses may also include an output that indicates the distraction state (e.g., thickness) of the apparatus. For example, any of these apparatuses may include visualization window showing an indicator of the current thickness. In some cases the visualization window may be positioned, e.g., through an anterior region of the femoral engagement plate, so that the visualization window allows visualization of the indicator. The location of the window on the anterior region of the femoral engagement plate may be particularly helpful, is it does not interfere with the contact between the femoral component and is visible for most of the time during the procedure. The window may allow viewing of a marking, which in some cases may be a distraction selector output indicator which may be on the distraction selector cam and may indicate the current distraction thickness between the femoral articular surface and the tibial seating surface.

Any of these apparatuses may also include a distraction input selector configured to be actuated to drive movement of the distraction selector cam to change the current distraction thickness between the femoral articular surface and the tibial seating surface. The distraction input selector may be configured as a mechanical selector, such as (but not limited to) a screw, key, rod, etc. In some cases the distraction input selector may be operated manually using a tool (such as a screwdriver, wrench, etc.).

The femoral engagement plate and the tibial plate engagement frame may generally be coupled together via one or more telescoping supports that allows the two to stably separate. In some cases the telescoping supports are configured to provide lateral support between the femoral articular surface and the tibial seating surface at each of the preset distraction thicknesses. For example, the telescoping supports may be positioned bilaterally on either side of a midline of the apparatus. The walls of the telescoping supports may mate with support walls that they telescope into and out of with a relatively tight tolerance (e.g., less than 0.8 mm, less than 0.5 mm, less than 0.3 mm, less than 0.2 mm, less than 0.1 mm, etc.). In some cases the apparatus, either part of the telescoping supports or separately, may include one or more biases (e.g. springs, compressible material, etc.) that may dampen or prevent instability. In some cases the apparatus may include one or more lateral supports to prevent twisting instability.

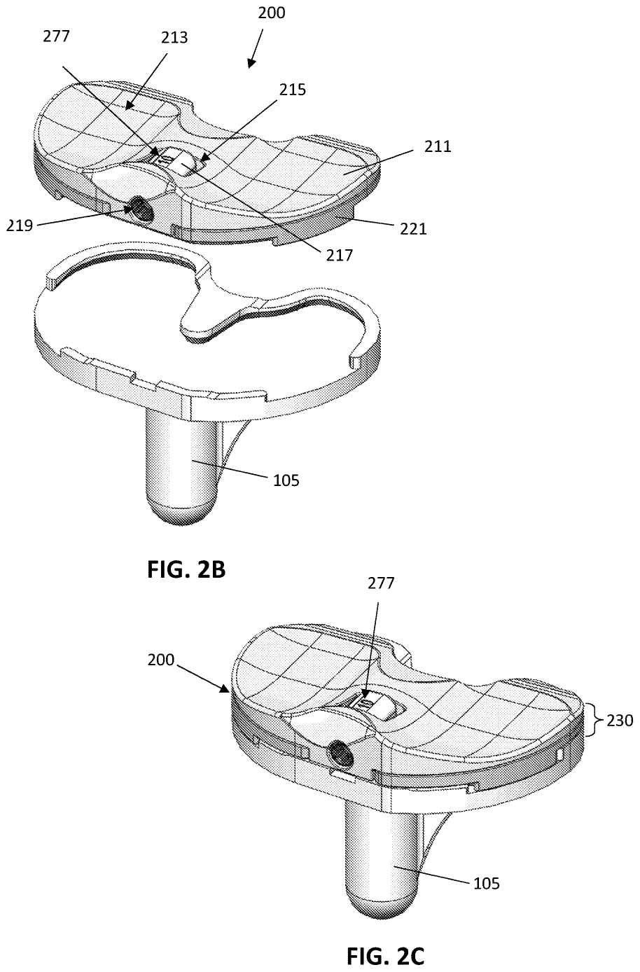

FIGS. 2A-2C schematically illustrates an example of an adjustable trialing tibial insert apparatus. For example, FIG. 2A shows an adjustable trialing tibial insert apparatus including a femoral engagement plate 211 having a femoral articular surface 213 that is configured to engage with a femoral component of a TKA system. The adjustable trialing tibial insert apparatus 200 also includes a tibial plate engagement frame 221 having a tibial seating surface 222 that is configured to engage with a tibial plate of the TKA system. The adjustable trialing tibial insert apparatus shown in FIGS. 2A-2C also includes a distraction selector cam 217 having a cam surface 267, the distraction selector cam has a plurality of flattened sides on the cam surface that each corresponding to a pre-set distraction thickness between the femoral articular surface and the tibial seating surface. In FIG. 2A, the distraction selector cam is visible through a visualization window 215, and the cam also includes a distraction selector output indicator 277, which in this example is shown as a numeric indicator of the thickness (e.g., 10 mm, 12 mm, 14, mm, etc.). In general, the adjustable trialing tibial insert apparatus may be configured to have any appropriate thickness, e.g., between 8 mm and 20 mm (including any increment therebetween, such as but not limited to: 8 mm, 9 mm, 10 mm, 11 mm, 12 mm, 13 mm, 14 mm, 15 mm, 16 mm, 17 mm, 18 mm, 19 mm, 20 mm, etc.). Any number of thickness settings may be used; in FIGS. 2A-2C, 3A-3B and 4A-4F, the three thickness settings are shown (10 mm, 12 mm and 14 mm).

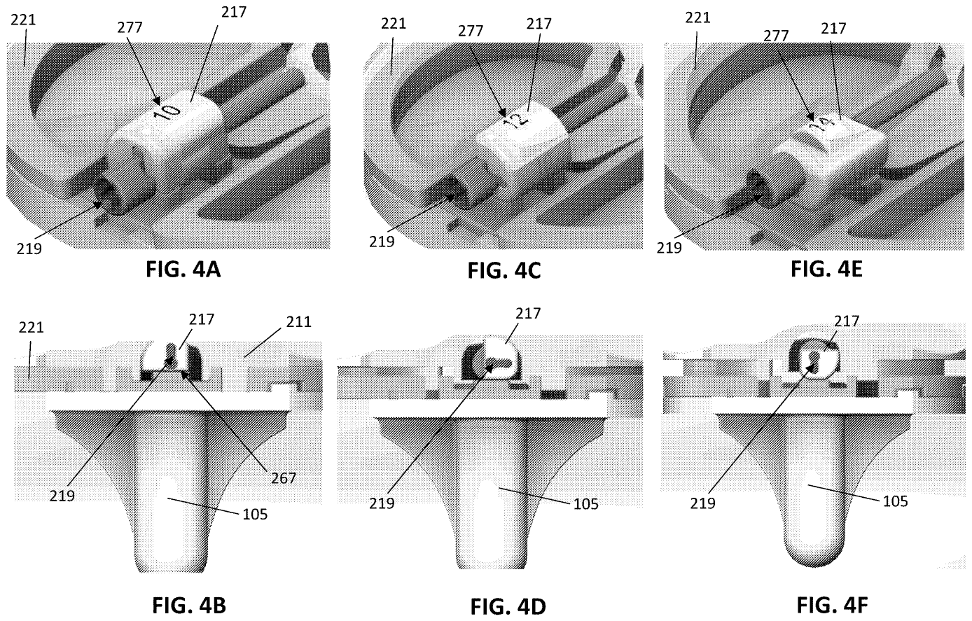

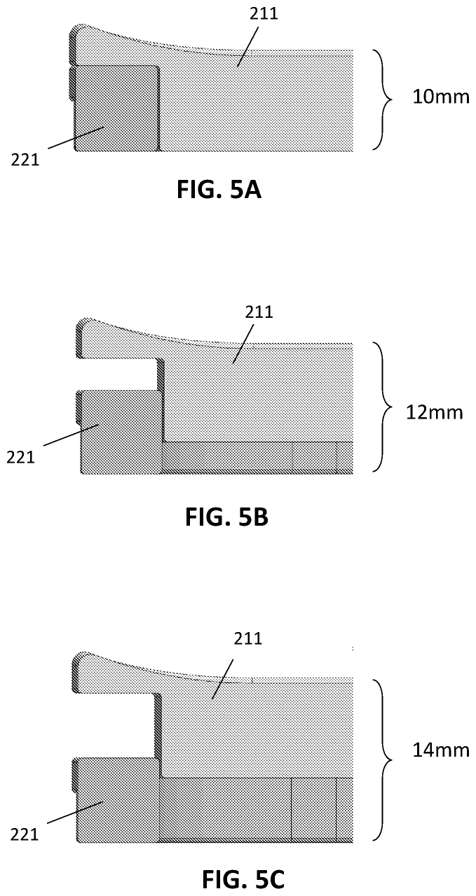

The adjustable trialing tibial insert apparatus in FIGS. 2A-2C thus includes a distraction input selector that is configured to be actuated by rotating (e.g., using a driver tool, not shown) to drive movement of the distraction selector cam to change the current distraction thickness between the femoral articular surface and the tibial seating surface. FIGS. 2A-2B illustrate the same adjustable trialing tibial insert apparatus 200 of FIG. 2A before (FIG. 2B) and after (FIG. 2C) seating on a stemmed tibial plate 105. In FIGS. 2A-2C the adjustable trialing tibial insert apparatus is shown at a minimum thickness 230, e.g., 10 mm. As will be illustrated in FIGS. 4A-4F and 5A-5C, this thickness may be changed.

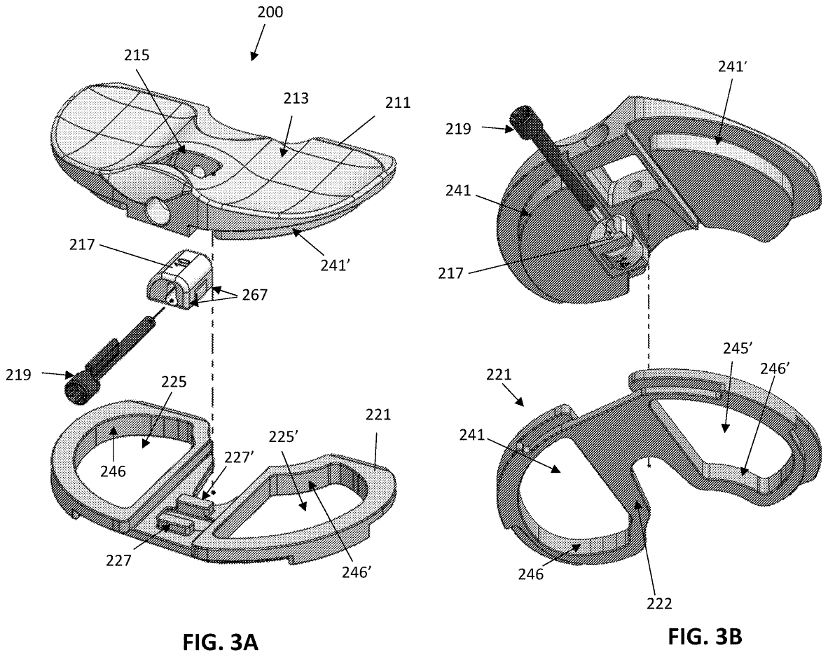

FIGS. 3A-3B and 4A-4F illustrate an adjustable trialing tibial insert apparatus similar to that shown in FIGS. 2A-2C. Similar numbers are used across the illustrations provided herein to denote elements having the same general functional characteristics, although different configurations and variations may be used. For example, FIGS. 3A-3B show front (anterior) perspective and back (posterior) perspective exploded views, respectively, of the adjustable trialing tibial insert apparatus of FIGS. 2A-2C. In FIG. 3A the femoral engagement plate 211 having a femoral articular surface 213 on the upper (outer) surface includes a visualization window 215 region cut through the femoral articular surface 213. The adjustable trialing tibial insert apparatus also include the distraction selector cam 217 that includes a cam surface 267. In this example the cam surface is divided over part of the cam circumference into two surfaces that both are configured to engage with distraction selector cam follower surfaces 227, 227′ that are configured to support the cam surface of the distraction selector cam. The distraction selector cam 217 is configured to be activated by rotating a distractor input selector 219. In this example the distractor input selector is shown as a keyed pin, which may engage with the cam 217 and drive the cam surface(s) against the follower surface(s) 227, 227′ to change the separation between the femoral engagement plate 211 and the tibial plate engagement frame 221.

As mentioned, the visualization window 215 passes through an anterior region of the femoral engagement plate 211, wherein the visualization window is configured allow visualization of a marking of the distraction selector output indicator 277 on the distraction selector cam 217 indicating the current thickness between the femoral articular surface and the tibial seating surface.

FIGS. 3A-3B also show a pair of telescoping support regions 241, 241′ (telescoping supports) that are bilaterally arranged on either side of the adjustable trialing tibial insert apparatus. In this example the femoral engagement plate and the tibial plate engagement frame are coupled together via these telescoping supports, so that as the separation between the femoral engagement plate and the tibial plate engagement frame changes (by moving the distraction selector cam), the telescoping supports extend 241, 241′ extend and/or retract relative to the telescoping support receiver regions (receivers) 225, 225′. This may provide lateral support between the femoral articular surface and the tibial seating surface at each of the preset distraction thicknesses. In this example, the telescoping supports are formed as part of the femoral engagement plate and the receivers are part of the tibial plate engagement frame 221, however, this arrangement may be reversed. The receivers include support walls 246, 246′ for supporting the telescoping support receiver. The receivers may be asymmetric in shape, which may increase the lateral support.

FIGS. 4A-4F illustrate distraction of an adjustable trialing tibial insert apparatus similar to that of FIGS. 2A-2C and 3A-3B. For example, FIGS. 4A-4B show front perspective and side views, respectively, of the adjustable trialing tibial insert apparatus in the most compact configuration (e.g., 10 mm). The distraction selector output indicator 277 is printed on the distraction selector cam 217, and is visible through the window through the femoral engagement plate. In FIGS. 4A and 4B, the distraction input selector 219 is shown with the notched, keyed region in the 12 o'clock position. The distraction selector cam has three a cam surface regions that are relatively flat and connected by curved regions. In FIGS. 4A-4B the flat region of the cam surface 267′ is engaged with the cam follower 227 and the spacing between the top surface and the bottom surface (e.g., the femoral articular surface and the tibial seating surface) is 10 mm.

FIGS. 4C and 4D show front perspective and side views, respectively, of the adjustable trialing tibial insert apparatus in a first pre-set thickness (e.g., 12 mm) configuration. In this example, the distraction input selector 219 is in the 3 o'clock position, and the eccentric outer cam surface has been rotated to a second stable position in which the relatively flat second region is engaged with the follower. In this case, the spacing between the top and bottom surfaces is 12 mm.

FIGS. 4E and 4F show front perspective and side views, respectively, of the adjustable trialing tibial insert apparatus in as second pre-set thickness (e.g., 14 mm) configuration. The distraction input selector 219 is in the 6 o'clock position, and the eccentric outer cam surface has been rotated to a third stable position in which the relatively flat third region is engaged with the cam follower. In this case, the spacing between the top and bottom surfaces is 14 mm. FIGS. 5A-5C show side sectional views through an adjustable trialing tibial insert apparatus similar to that shown in FIGS. 4A-4F, illustrating the change in distraction (thickness) of the femoral articular surface of the femoral engagement plate 211 relative to the tibial plate engagement frame 221. In FIG. 5A the separation is 10 mm, in FIG. 5B the separation is 12 mm and in FIG. 5C the separation is 14 mm.

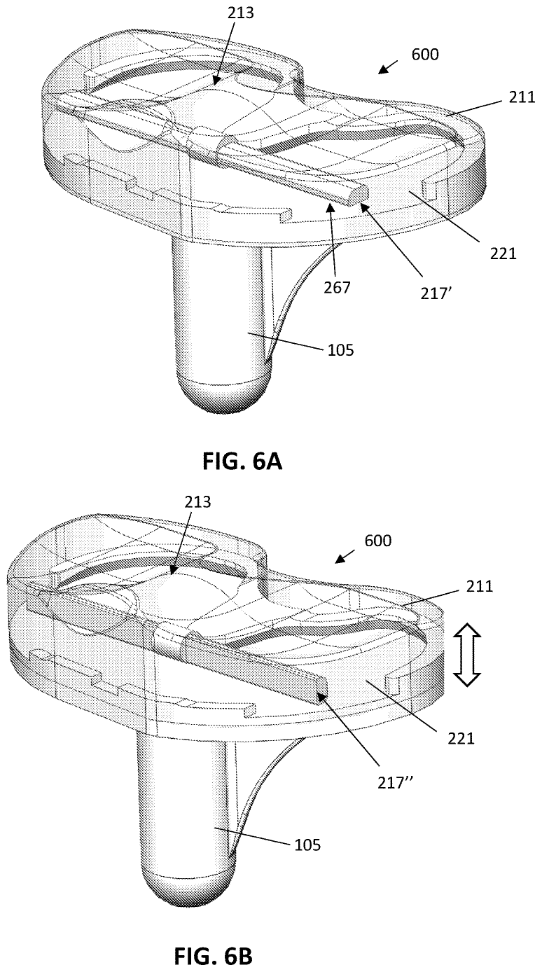

FIGS. 6A-6B illustrate a second example of an adjustable trialing tibial insert apparatus 600 described herein. This configuration is similar to that shown in FIGS. 2A-2C, 3A-3B, 4A-4F and 5A-5C, but the distraction selector cam 217′, 217″ is arranged along the long axis (e.g., transverse to the Anterior-Posterior axis) of the adjustable trialing tibial insert apparatus. This adjustable trialing tibial insert apparatus also includes a femoral engagement plate 211 having a femoral articular surface 213 that is configured to engage with a femoral component of a TKA system and a tibial plate engagement frame 221 having a tibial seating surface that is configured to engage with a tibial plate of the TKA system. In this example the cam 217′ may be rotated (e.g., 90 degrees 217″) to increase the separation between the femoral articular surface and the tibial seating surface, e.g., 2 mm. FIG. 6A shows the device having a 10 mm thickness and FIG. 6B shows the device with a 12 mm thickens.

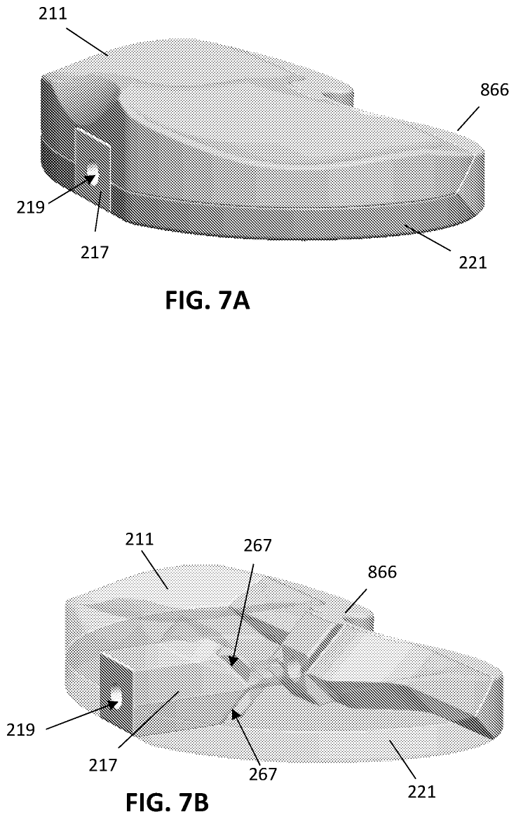

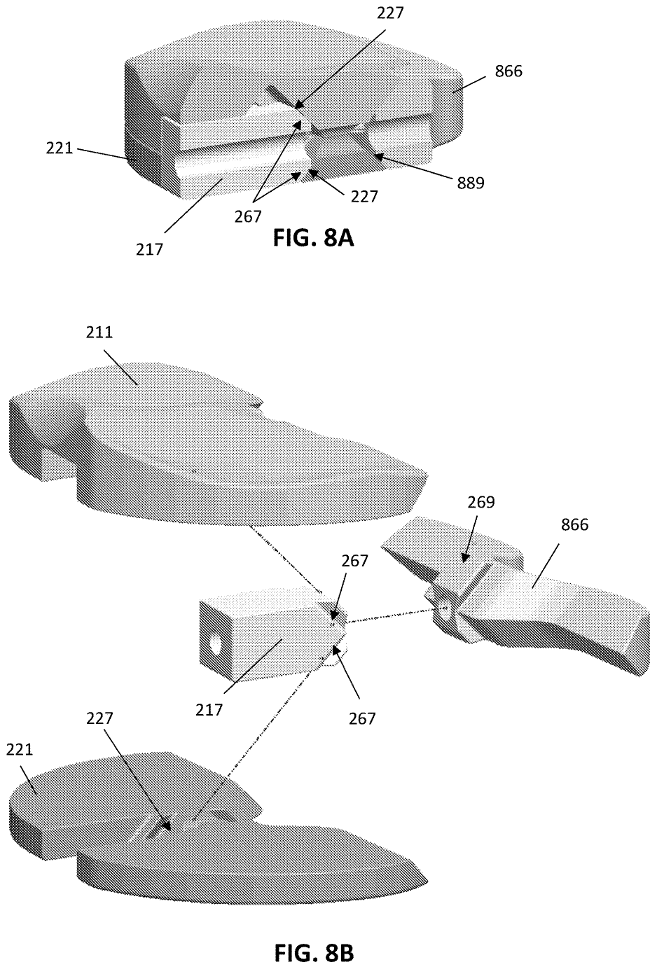

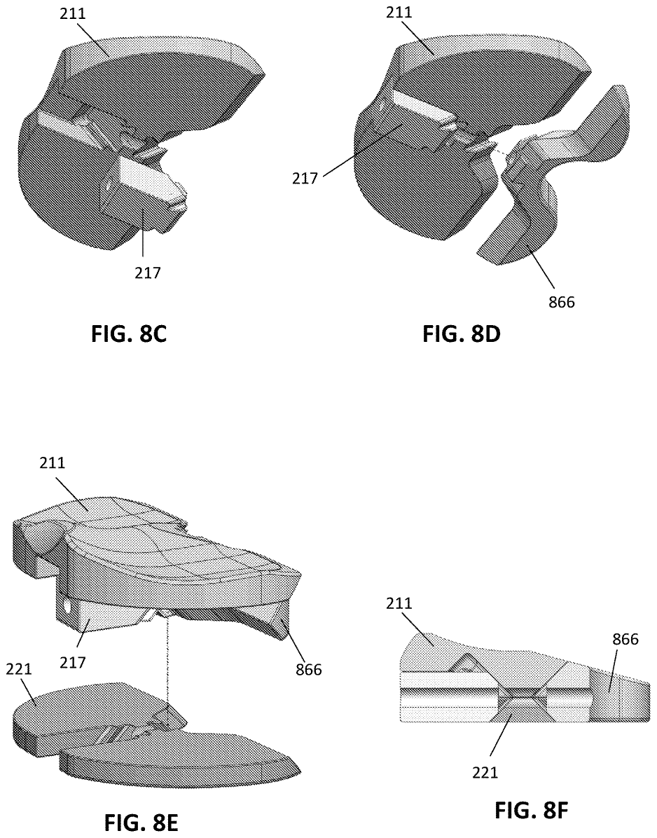

FIGS. 7A-7B schematically illustrate another example of an adjustable trialing tibial insert apparatus described herein. FIG. 7A shows a side perspective view and FIG. 7B shows a partially transparent view. In this example a pair of cam elements, anterior and posterior, may be used to change the separation between the femoral articular surface and the tibial seating surface; a distraction input selector (such as those shown above, and/or a threaded distraction input selector) may be used to change the relative separation between the anterior distraction selector cam 217 and a posterior cam member (posterior cam) 866. The device may include additional cam follower surfaces, as illustrated in FIGS. 7B and 8A-8F. In FIGS. 7A-7B the adjustable trialing tibial insert apparatus includes a tibial plate engagement frame 221 and a femoral engagement frame 211 as well as the anterior distractor cam 217 and posterior cam 866. In this example, both the tibial plate engagement frame 221 and the femoral engagement plate 211 include follower surfaces, e.g., distraction selector cam followers 227 and posterior cam followers 889.

FIGS. 8A-8F show additional views of the adjustable trialing tibial insert apparatus of FIGS. 7A-7B. FIG. 8A shows a section through the anterior-to-posterior axis (midline) of the adjustable trialing tibial insert apparatus. FIG. 8B shows an exploded view. FIGS. 8C and 8D show bottom perspective views of a portion of the adjustable trialing tibial insert apparatus. FIG. 8E shows a partially exploded view of the adjustable trialing tibial insert apparatus. FIG. 8F shows a section through the midline of the adjustable trialing tibial insert apparatus.

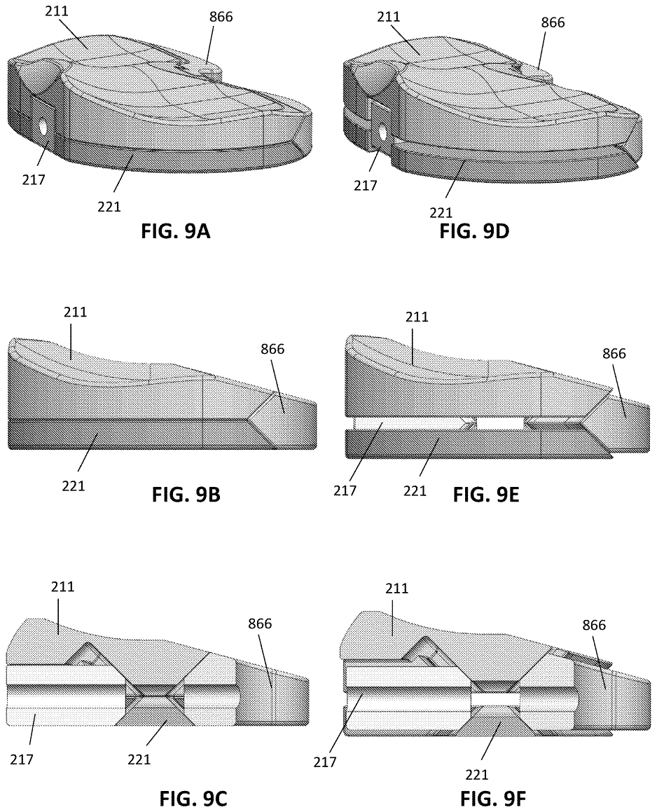

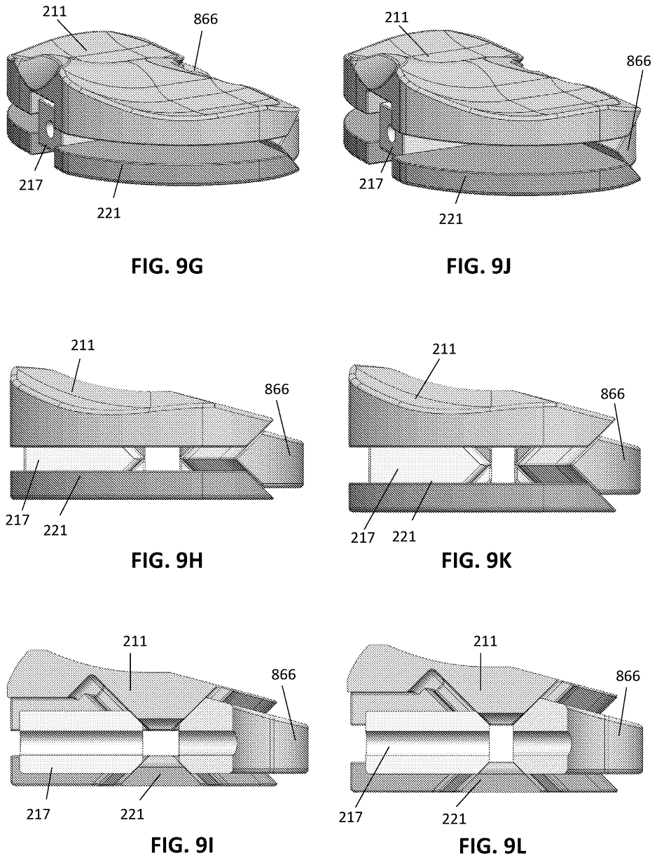

FIGS. 9A-9L illustrate distraction (e.g., controllably setting the thickness) of the adjustable trialing tibial insert apparatus described in FIGS. 7A-7B and 8A-8F. For example, FIGS. 9A, 9B and 9C show the adjustable trialing tibial insert apparatus in a first thickness configuration (e.g., 10 mm separation between the femoral articular surface and the tibial seating surface), in a side perspective, side and sectioned views, respectively. FIGS. 9D, 9E and 9F show the adjustable trialing tibial insert apparatus in a second thickness configuration (e.g., 12 mm separation between the femoral articular surface and the tibial seating surface), in a side perspective, side and sectioned views, respectively. FIGS. 9G, 9H and 9I show the adjustable trialing tibial insert apparatus in a third thickness configuration (e.g., 14 mm separation between the femoral articular surface and the tibial seating surface), in a side perspective, side and sectioned views, respectively. And FIGS. 9J, 9K and 9L show the adjustable trialing tibial insert apparatus in a fourth thickness configuration (e.g., 16 mm separation between the femoral articular surface and the tibial seating surface), in aside perspective, side and sectioned views, respectively. In FIGS. 9A-9L, as the distraction input selector is actuated, the distraction selector cam (e.g., anterior distraction selector cam) and the posterior distraction selector cam are moved relative to each other to change the separation between the top and bottom of the adjustable trialing tibial insert apparatus. Thus, activation of the distraction input selector (not shown in FIGS. 9A-9L for simplicity) changes the distance between the posterior cam member and the (anterior) distraction selector cam to change the distraction thickness between the femoral articular surface and the tibial seating surface.

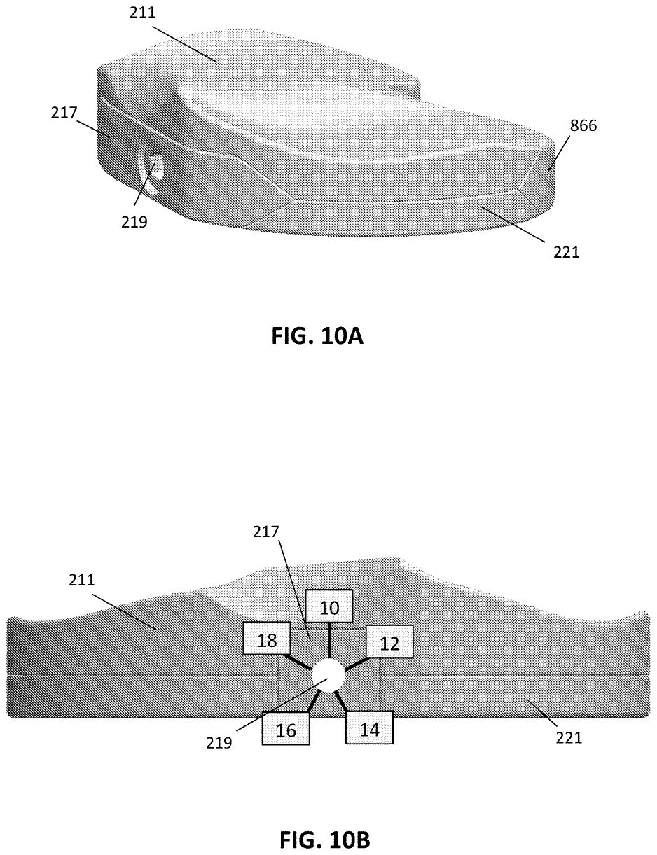

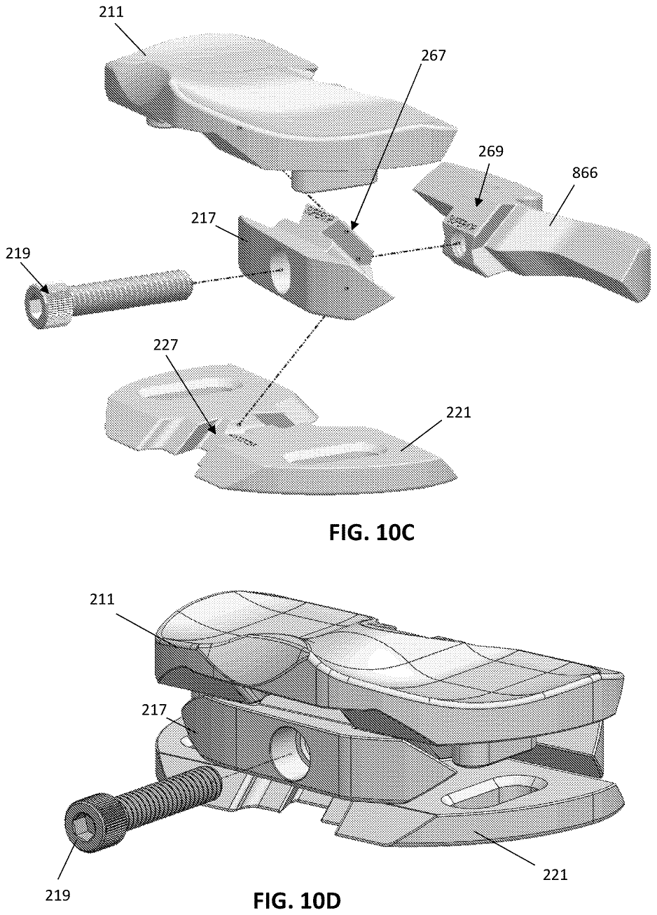

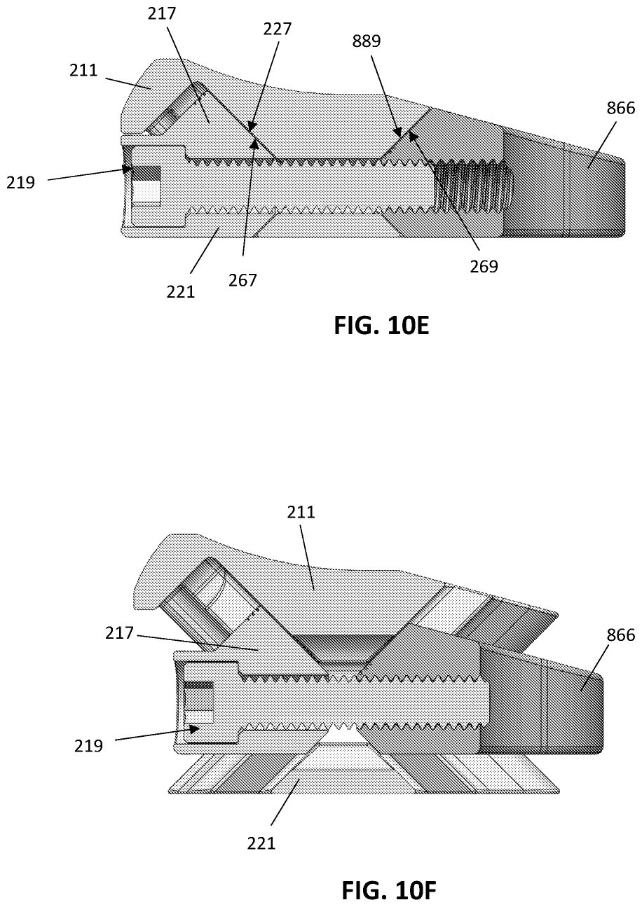

FIGS. 10A-10F schematically illustrate another example of an adjustable trialing tibial insert apparatus as described herein, similar to that shown in FIGS. 7A-7B, 8A-8F and 9A-9L. FIG. 10A shows a side perspective view of the device, which also includes a femoral engagement plate 211 having a femoral articular surface that is configured to engage with a femoral component of a TKA system and a tibial plate engagement frame 221 having a tibial seating surface that is configured to engage with a tibial plate of the TKA system. The apparatus also includes a distractor selector cam (shown as an anterior cam 217) and a distraction input selector 219, as well as a posterior cam 866. As shown in FIG. 10B the device is configured to have five distraction settings (e.g., 10 mm, 12 mm, 14 mm, 16 mm and 18 mm). The distraction input selector may therefore be configured to switch between these five states. FIG. 10C shows an exploded view of this apparatus. Including the distraction input selector, which is configured as a threaded screw 219. The cam surface 267 of the distraction selector cam 217 has a dovetail configuration. FIG. 10D shows an anterior perspective view of the assembled apparatus of FIGS. 10A-10C. FIGS. 10E-10F show section views through a midline of the adjustable trialing tibial insert apparatus, illustrating operation of this example. In FIG. 10E the threaded distraction input selector is withdrawn, and the apparatus has its lowest thickness (e.g., 10 mm). In FIG. 10F, the distraction input selector 219 is advanced (e.g., by turning the screw-like body) so that the femoral engagement plate 211 is separated from the tibial plate engagement frame 221 (e.g., having an 18 mm separation).

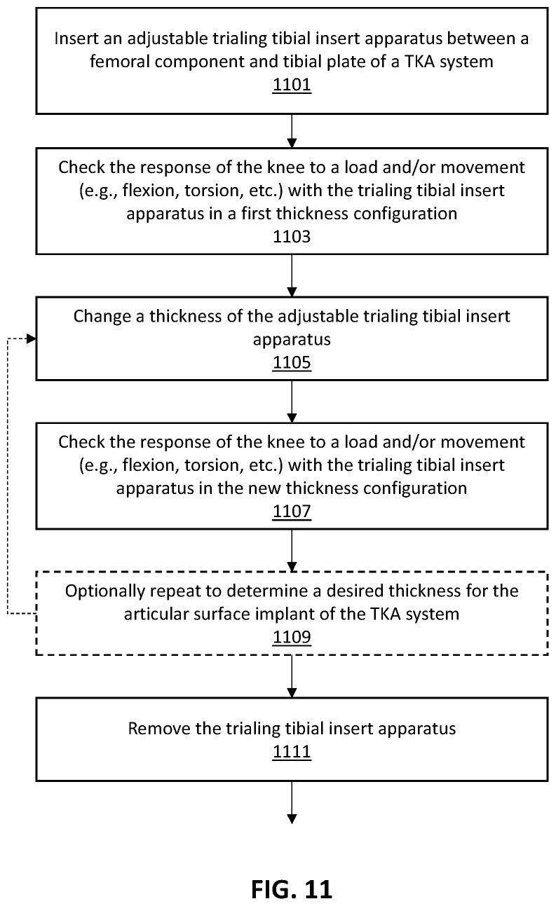

In use, this apparatus may be configured to be part of a TKI procedure, as mentioned above. For example, FIG. 11 schematically illustrates one example of a method of using an adjustable trialing tibial insert apparatus as described herein as part of a TKI procedure. In FIG. 11, the method may be a method of determining an articular surface thickness for a total knee arthroplasty (TKA) or revision procedure. The adjustable trialing tibial insert apparatus may be inserted 1101 after the TKA system has been attached to the patient. For example, an adjustable trialing tibial insert apparatus as described herein may be inserted between a femoral component and tibial plate of a TKA system, wherein the adjustable trialing tibial insert is in a first thickness configuration. The adjustable trialing tibial insert apparatus, once seated on the tibial plate of the TKA system, may then be iteratively adjusted to different thicknesses and the knee joint examined by the user to determine an optimal thickness for the implant that will replace the adjustable trialing tibial insert apparatus. For example, the user may check the response of the knee to a load and/or movement with the adjustable trialing tibial insert apparatus in a first thickness configuration 1103. The user may then change the thickness of the adjustable trialing tibial insert apparatus from the first thickness configuration to a second, pre-set, thickness configuration 1105 and check the response of the knee to the second thickness. The user may test the response of the knee with a variety of different tests, including loading/unloading the knee, and/or a plurality of flexion and extension cycles and/or torsional/twisting movement. After each change in the thickness, the knee may be tested 1107. This process may be repeated 1109 as much as desired until the user determines which thickness is optimal. After this, the user may remove the adjustable trialing tibial insert apparatus 1111. An articular surface to be positioned between the femoral component and tibial plate of the TKA system may then be selected and inserted based on the responses of the knee to the load and/or movement at the different thicknesses tested.

In any of these methods an apparatus an additional stabilization piece may be applied, e.g., at least partially around the perimeter of the apparatus after adjusting the height. For example, an outer U-shaped member may fit around the perimeter of the apparatus at each or every thickness, which may enhance stability. This U-shaped member may releasably engage with the side of the apparatus.

These apparatuses may be easily disassembled for cleaning and sterilization purposes. In all designs the base or lower part of the construct will sit within the tibial component with appropriate locking and will have an upper part that has articular geometry to match the femoral component.

All publications and patent applications mentioned in this specification are herein incorporated by reference in their entirety to the same extent as if each individual publication or patent application was specifically and individually indicated to be incorporated by reference. Furthermore, it should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under”, or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An adjustable trialing tibial insert apparatus for setting articular surface thickness during a total knee arthroplasty (TKA) or revision procedure, the apparatus comprising:
a femoral engagement plate having a femoral articular surface that is configured to engage with a femoral component of a TKA system;
a tibial plate engagement frame having a tibial seating surface that is configured to engage with a tibial plate of the TKA system;
a distraction selector cam having a cam surface, the cam surface a plurality of positions comprising a plurality of pre-set distraction thickness between the femoral articular surface and the tibial seating surface; and
a distraction input selector coupled to the distraction selector cam and configured to be actuated to drive movement of the distraction selector cam against to change a distraction thickness between the femoral articular surface and the tibial seating surface.

2. The apparatus of claim 1, wherein the cam surface comprises a plurality of flattened sides each corresponding to a pre-set distraction thickness of the plurality of pre-set distraction thicknesses between the femoral articular surface and the tibial seating surface.

3. The apparatus of claim 1, further comprising a visualization window through the femoral engagement plate, wherein the visualization window is configured allow visualization of a distraction selector output indicator indicating a current distraction thickness between the femoral articular surface and the tibial seating surface.

4. The apparatus of claim 3, wherein the visualization window is on a central anterior region of the femoral engagement plate.

5. The apparatus of claim 3, wherein the distraction selector output indicator is indicating the pre-set distraction thickness between the femoral articular surface and the tibial seating surface is on the distraction selector cam.

6. The apparatus of claim 1, wherein the plurality of pre-set distraction thicknesses correspond to thickness between 8 mm and 16 mm.

7. The apparatus of claim 1, wherein the plurality of pre-set distraction thicknesses include at least: 9 mm, 11 mm, and 13 mm.

8. The apparatus of claim 1, wherein the plurality of pre-set distraction thicknesses include at least: 10 mm, 12 mm and 14 mm.

9. The apparatus of claim 1, further comprising one or more selector cam follower surfaces.

10. The apparatus of claim 9, wherein the one or more selector cam follower surfaces are on the tibial seating surface.

11. The apparatus of claim 1, wherein the distraction input selector is on an anterior region of the apparatus.

12. The apparatus of claim 1, wherein the femoral engagement plate and the tibial plate engagement frame are coupled together via one or more telescoping supports configured to provide lateral support and prevent rocking between the femoral articular surface and the tibial seating surface at each of the preset distraction thicknesses corresponding to the plurality of flattened sides on the cam surface.

13. The apparatus of claim 12, wherein the one or more telescoping supports comprises a plurality of telescoping supports.

14. The apparatus of claim 12, wherein the one or more telescoping supports comprises a plurality of bilateral telescoping supports.

15. The apparatus of claim 12, further comprising a posterior cam member coupled to the distraction selector cam via the distraction input selector, wherein activation of the distraction input selector changes the distance between the posterior cam member and the distraction selector cam to change the distraction thickness between the femoral articular surface and the tibial seating surface.

16. An adjustable trialing tibial insert apparatus for setting articular surface thickness during a total knee arthroplasty (TKA) or revision procedure, the apparatus comprising:
a femoral engagement plate having a femoral articular surface that is configured to engage with a femoral component of a TKA system;
a tibial plate engagement frame having a tibial seating surface that is configured to engage with a tibial plate of the TKA system;
a distraction selector cam having a cam surface, the distraction selector cam comprising a plurality of flattened sides on the cam surface each corresponding to a pre-set distraction thickness between the femoral articular surface and the tibial seating surface;
one or more distraction selector cam follower surfaces configured to support the cam surface of the distraction selector cam; and
a distraction input selector coupled to the distraction selector cam and configured to be actuated to drive movement of the distraction selector cam against the one or more distraction selector cam follower surfaces to change a distraction thickness between the femoral articular surface and the tibial seating surface;
wherein the femoral engagement plate and the tibial plate engagement frame are coupled together via one or more telescoping supports configured to provide lateral support and prevent rocking at each of the preset distraction thicknesses corresponding to the plurality of flattened sides on the cam surface.

17. An adjustable trialing tibial insert apparatus for setting articular surface thickness during a total knee arthroplasty (TKA) or revision procedure, the apparatus comprising:
a femoral engagement plate having a femoral articular surface that is configured to engage with a femoral component of a TKA system;
a tibial plate engagement frame having a tibial seating surface that is configured to engage with a tibial plate of the TKA system;
a distraction selector cam having a cam surface, the distraction selector cam comprising a plurality of flattened sides on the cam surface each corresponding to a pre-set distraction thickness between the femoral articular surface and the tibial seating surface;
one or more distraction selector cam follower surfaces configured to support the cam surface of the distraction selector cam;
a visualization window through an anterior region of the femoral engagement plate, wherein the visualization window is configured allow visualization of a distraction selector output indicator on the distraction selector cam indicating a current distraction thickness between the femoral articular surface and the tibial seating surface;
a distraction input selector configured to be actuated to drive movement of the distraction selector cam to change the current distraction thickness between the femoral articular surface and the tibial seating surface,
wherein the femoral engagement plate and the tibial plate engagement frame are coupled together via one or more telescoping supports configured to provide lateral support between the femoral articular surface and the tibial seating surface at each of the preset distraction thicknesses.

18. A method of determining an articular surface thickness for a total knee arthroplasty (TKA) or revision procedure using an adjustable trialing tibial insert apparatus, the method comprising:
inserting an adjustable trialing tibial insert apparatus between a femoral component and tibial plate of a TKA system, wherein the adjustable trialing tibial insert is in a first thickness configuration;
checking a response of the knee to a load and/or movement corresponding to the first thickness configuration;
changing the thickness of the adjustable trialing tibial insert apparatus from the first thickness configuration to a second, pre-set, thickness configuration;
checking a response of the knee to a second load and/or movement corresponding to the second, pre-set, thickness configuration; and
removing the adjustable trialing tibial insert apparatus and selecting an articular surface to be positioned between the femoral component and tibial plate of the TKA system based on the responses of the knee to the load and/or movement.

19. The method of claim 18, further comprising repeating the steps of changing the thickness of the adjustable trialing tibial insert apparatus and checking the response of the knee before removing the adjustable trialing tibial insert.

20. The method of claim 18, wherein checking the response of the knee to the load and/or movement comprises checking the response of the knee to one or more of: a plurality of flexion and extension cycles and a torsional/twisting movement.

21. The method of claim 18, wherein changing the thickness of the adjustable trialing tibial insert apparatus from the first thickness configuration to the second, pre-set, thickness configuration comprises activating a distraction input selector to drive movement of a distraction selector cam against one or more distraction selector cam follower surfaces to change a distraction thickness between a femoral articular surface and a tibial seating surface of the adjustable trialing tibial insert apparatus.

22. The method of claim 21, wherein changing the thickness of the adjustable trailing tibial insert apparatus comprises rotating the distraction selector cam so that one of a plurality of flattened sides on the cam surface rests against the one or more distraction selector cam follower surfaces, wherein each of flattened side of the plurality of flattened sides corresponds to a pre-set distraction thickness between the femoral articular surface and the tibial seating surface.

23. The method of claim 18, further comprising visually observing a distraction selector output indicator through a visualization window of a femoral articular surface of the adjustable trialing tibial insert apparatus.
